(19) United States
(12) Patent Application Publication
(10) Pub. No.: US 2024/0158754 A1
(43) Pub. Date: May 16, 2024

(54) REDUCING HIGH MANNOSE GLYCAN PROTEIN EXPRESSION USING GUANOSINE 5'-MONOPHOSPHATE

(71) Applicant: MERCK SHARP & DOHME LLC, Rahway, NJ (US)

(72) Inventors: Gaurav Chauhan, Downingtown, PA (US); Wai Lam Wong Ling, East Brunswick, NJ (US)

(73) Assignee: MERCK SHARP & DOHME LLC, Rahway, NJ (US)

(21) Appl. No.: 18/548,640

(22) PCT Filed: Mar. 3, 2022
(86) PCT No.: PCT/US2022/018607

§ 371 Date: Sep. 1, 2023

Related U.S. Application Data
(60) Provisional application No. 63/157,902, filed on Mar. 8, 2021.

(51) Int. Cl. C12N 5/071

(52) U.S. Cl. CPC C12N 5/0682; C12N 2500/40

(57) ABSTRACT

The present invention provides a method of reducing a high mannose glycan (HMG) content of a protein (e.g., monoclonal antibody) expressed during a mammalian cell culture (e.g., CHO cell culture) process, as well as a cell culture medium for reducing an HMG content of a protein (e.g., monoclonal antibody) expressed during a mammalian cell culture (e.g., CHO cell culture) process.

### FIELD OF THE INVENTION

Provided herein are methods of reducing high mannose glycan (HMG) content of proteins (e.g., monoclonal antibodies) expressed in cell cultures (e.g., CHO cell cultures) as well as cell culture media for achieving such goals.

### CROSS-REFERENCE TO RELATED APPLICATIONS

This international application claims the benefit of priority to U.S. Provisional Application No. 63/157,902, filed Mar. 8, 2021, the entirety of which is incorporated herein by reference.

### BACKGROUND OF THE INVENTION

Appropriate glycosylation is critical for the conformation, solubility, stability, antigenicity, and in vivo efficacy of therapeutic proteins. N-glycosylation of proteins are classified into 3 general forms (high mannose type, complex type, and hybrid type) (Park and Lee, Gut and Liver, 2013, 7(6):629-641). Tissue culture systems (e.g., CHO cells) frequently produce the high mannose type. High mannose glycan (HMG), with 5-9 mannose attached sugars, can directly impact the clearance rate of the therapeutic proteins, hence affecting the pharmacokinetics of the therapeutic proteins (Goetze et al., Glycobiology, 2011, 21(7):949-959). HMG can also enhance antibody-dependent cell-mediated cytotoxicity (ADCC) compared with antibodies with fucosylated complex or hybrid glycans (Yu, et al., MAbs, 2012, 4:475-87). The presence of the HMG may also impact immunogenicity (Arnold et al., Glycobiology and Medicine (Axford ed.), 2007, 27-43). Thus, glycoengineering and glycosylation control during bioprocess development are rational strategies to improve safety and efficacy of the therapeutic proteins.

Methods for manipulating HMG content of a protein in cell culture include changes in media compositions, osmolality, pH, temperature, etc. (Yu, et al., supra; Pacis et al., Biotechnol Bioeng, 2011, 108, 2348-2358; Chee Furng Wong et al., Biotechnol Bioeng, 2005, 89:164-177; Ahn, et al., Biotechnol Bioeng, 2008, 101:1234-44). The effectiveness of these methods is specific to cell lines, molecule types, and media environment, and is typically obtained by trial and error. Additionally, these methods tend to also alter productivity, cell culture parameters, and other protein attributes.

Thus, there remain unmet needs to effectively reduce HMG content in proteins (e.g., monoclonal antibodies) expressed in cell cultures (e.g., CHO cells) without compromising production culture performance and yield.

### SUMMARY OF THE INVENTION

The present invention provides a method of reducing a high mannose glycan (HMG) content of a protein (e.g., monoclonal antibody) expressed during a mammalian cell culture (e.g., CHO cell culture) process, as well as a cell culture medium for reducing an HMG content of a protein (e.g., monoclonal antibody) expressed during a mammalian cell culture (e.g., CHO cell culture) process.

In one aspect, provided is a method of reducing a HMG content of a protein expressed during a mammalian cell culture process, comprising:
(1) establishing a mammalian cell culture expressing the protein; and
(2) contacting the cell culture with Guanosine 5′-monophosphate (GMP) or an agent that increases GMP de novo synthesis.

In certain embodiments of the method, step (2) occurs during a growth phase of the cell culture.

In some embodiments of the method, step (2) occurs during a production phase of the cell culture.

In other embodiments of the method, step (2) occurs during a growth phase and a production phase of the cell culture.

In some embodiments of various methods described herein, step (2) comprises contacting the cell culture with GMP.

In other embodiments of various methods described herein, step (2) comprises contacting the cell culture with an agent that increases GMP de novo synthesis.

In various embodiments, the agent that increases GMP de novo synthesis is Inosine 5′-monophosphate (IMP), Xanthine 5′-monophosphate (XMP), Guanosine 5′-diphosphate (GDP), Guanosine 5′-triphosphate (GTP), Guanosine 3′,5′-cyclic monophosphate (cGMP), Guanine, IMP dehydrogenase, or GMP synthase. In one embodiment, the agent is IMP. In another embodiment, the agent is XMP. In yet another embodiment, the agent is GDP. In still another embodiment, the agent is GTP. In yet still another embodiment, the agent is cGMP. In one embodiment, the agent is Guanine. In another embodiment, the agent is IMP dehydrogenase. In yet another embodiment, the agent is GMP synthase.

In certain embodiments of the various methods described herein, the GMP is present in the cell culture for a time period of about 1, 2, 3, 4, 5, 6, 7, 8, 9, or 10 days or longer. In certain embodiments of the various methods described herein, the GMP is present in the cell culture for a time period of 1, 2, 3, 4, 5, 6, 7, 8, 9, or 10 days or longer.

In other embodiments, the GMP is present in the cell culture for the entire duration of the cell culture.

In some embodiments, the final concentration of GMP in the cell culture is from 1 mM to 25 mM.

In certain embodiments, the final concentration of GMP in the cell culture is from 1 mM to 20 mM, from 1 mM to 3 mM, from 2 mM to 5 mM, from 3 mM to 20 mM, from 3 mM to 18 mM, from 3 mM to 15 mM, from 5 mM to 15 mM, or from 3 mM to 10 mM.

In other embodiments, the final concentration of GMP in the cell culture is 1 mM, 3 mM, 5 mM, 10 mM, 15 mM, 18 mM, or 20 mM. In one embodiment, the final concentration of GMP in the cell culture is 1 mM. In another embodiment, the final concentration of GMP in the cell culture is 10 mM.

In some embodiments, the GMP is added to the cell culture between 3 and 15 days after the cell culture is established.

In certain embodiments, the GMP is added to the cell culture at about day 3, at about day 4, at about day 5, at about day 6, at about day 7, at about day 8, at about day 9, at about day 10, at about day 11, or at about day 12 after the cell culture is established. In certain embodiments, the GMP is added to the cell culture at day 3, at day 4, at day 5, at day 6, at day 7, at day 8, at day 9, at day 10, at day 11, or at day 12 after the cell culture is established. In one embodiment, the GMP is added to the cell culture at about day 6 after the cell culture is established.

In some embodiments of various methods described herein, the cell culture is maintained by perfusion for a partial or entire period of the cell culture. In one embodiment, the perfusion begins on day 1 of the cell culture. In another embodiment, the perfusion begins when the cell culture reaches a production phase.

In certain embodiments of various methods described herein, the cell culture is maintained by fed batch for a partial or entire period of the cell culture. In one embodiment, the fed batch begins on day 1 of the cell culture. In another embodiment, the fed batch begins when the cell culture reaches a production phase.

In other embodiments of various methods described herein, the cell culture is maintained by a combination of fed batch and perfusion for a partial or entire period of the cell culture. In some embodiments, the cell culture is maintained by fed batch in the growth phase and by perfusion in the production phase. In one embodiment, perfusion begins before the cell culture reaches the production phase. In another embodiment, perfusion begins at or about the time when the cell culture reaches the production phase. In yet another embodiment, perfusion begins after the cell culture reaches the production phase.

In some embodiments, the HMG content of the protein is reduced by at least 1%, 3%, 5%, 10%, 15%, 20%, 25%, or 30% compared to the HMG content of the protein expressed in an essentially same cell culture except that the essentially same cell culture is not contacted with GMP or the agent that increases GMP de novo synthesis. In one embodiment, the HMG content of the protein is reduced by at least 3%. In another embodiment, the HMG content of the protein is reduced by at least 5%. In yet another embodiment, the HMG content of the protein is reduced by at least 10%. In still another embodiment, the HMG content of the protein is reduced by at least 20%. In yet still another embodiment, the HMG content of the protein is reduced by at least 30%.

In certain embodiments, the HMG is Man 5.

In some embodiments of various methods described herein, the method further comprises a harvest step.

In another aspect, provided is a cell culture medium for reducing a HMG content of a protein expressed during a mammalian cell culture process, comprising GMP or an agent that increases GMP de novo synthesis.

In certain embodiments of various media described herein, the GMP or the agent that increases GMP de novo synthesis is at a final concentration that is sufficient to reduce the HMG content of the protein by at least 1%, 3%, 5%, 10%, 15%, 20%, 25%, or 30% compared to the protein expressed in an essentially same cell culture medium except that the essentially same cell culture medium does not include the GMP or the agent that increases GMP de novo synthesis.

In one embodiment, the cell culture medium comprises GMP. In some embodiments, the GMP is at a final concentration of 1-25 mM. In certain embodiments, the GMP is at a final concentration of 1-20 mM, 3-20 mM, 3-18 mM, 3-15 mM, 5-15 mM, or 3-10 mM. In other embodiments, the GMP is at a final concentration of 1 mM, 3 mM, 5 mM, 10 mM, 15 mM, 18 mM, or 20 mM. In one specific embodiment, the GMP is at a final concentration of 1 mM. In another specific embodiment, the GMP is at a final concentration of 10 mM.

In another embodiment, the cell culture medium comprises an agent that increases GMP de novo synthesis. In various embodiments, the agent is Inosine 5′-monophosphate (IMP), Xanthine 5′-monophosphate (XMP), Guanosine 5′-diphosphate (GDP), Guanosine 5′-triphosphate (GTP), Guanosine 3′,5′-cyclic monophosphate (cGMP), Guanine, IMP dehydrogenase, or GMP synthase. In one embodiment, the agent is IMP. In another embodiment, the agent is XMP. In yet another embodiment, the agent is GDP. In still another embodiment, the agent is GTP. In yet still another embodiment, the agent is cGMP. In one embodiment, the agent is Guanine. In another embodiment, the agent is IMP dehydrogenase. In yet another embodiment, the agent is GMP synthase.

In certain embodiments of methods and cell culture media described herein, the mammalian cell culture is a CHO cell culture. In some embodiments, the cell culture medium is a CHO cell culture medium.

In various embodiments of methods and cell culture media described herein, the protein is a therapeutic protein, monoclonal antibody, hormone, cytokine, growth factor, clotting factor, enzyme, fusion protein thereof, immunoconjugate thereof, or fragment thereof. In certain embodiments, the protein is a therapeutic protein. In some embodiments, the protein is a monoclonal antibody. In other embodiments, the protein is a hormone. In yet other embodiments, the protein is a cytokine. In still other embodiments, the protein is a growth factor. In certain embodiments, the protein is a clotting factor. In some embodiments, the protein is an enzyme. In other embodiments, the protein is a fusion protein of the above proteins. In yet other embodiments, the protein is an immunoconjugate of the above proteins. In still other embodiments, the protein is a fragment of the above proteins.

### BRIEF DESCRIPTION OF THE DRAWINGS

FIG. 1 illustrates exemplary GMP de novo synthesis pathways.

FIGS. 2A-2C show that various components added to cell culture media exhibited different effects on HMG content and quality attributes of mAb A (FIG. 2A), mAb B (FIG. 2B), or mAb C (FIG. 2C) expressed in CHO cell lines.

FIG. 3 shows that GMP caused a dose dependent decrease in Man 5 content on three different antibodies (mAb A, mAb B, and mAb C) expressed in various CHO cell lines.

FIGS. 4A-4E demonstrate that GMP did not significantly affect cell culture performance parameters of three CHO cell lines expressing mAb A, mAb B, or mAb C: integral viable cell concentration (IVCC) (FIG. 4A), final viability (FIG. 4B), titer (FIG. 4C), ammonia (FIG. 4D), and lactate (FIG. 4E).

FIGS. 5A-5B demonstrate the effect of GMP on the content of high molecular weight (HMW) (FIG. 5A) or monomer (FIG. 5B) species of mAb A, mAb B, or mAb C.

FIGS. 6A-6C demonstrate the effect of GMP on acidic (FIG. 6A), main (FIG. 6A), or basic (FIG. 6C) charge variant species of mAb A, mAb B, and mAb C.

### DETAILED DESCRIPTION OF THE INVENTION

The present invention provides a method of reducing a high mannose glycan (HMG) content of a protein (e.g., monoclonal antibody) expressed during a mammalian cell culture (e.g., CHO cell culture) process, as well as a cell culture medium for reducing an HMG content of a protein (e.g., monoclonal antibody) expressed during a mammalian cell culture (e.g., CHO cell culture) process.

Eighteen different components that supplement cell culture media were screened for their effects on three monoclonal antibodies (mAb A, mAb B, and mAb C) expressed in different CHO cell lines. Among the 18 tested components, guanosine 5′-monophosphate (GMP), also referred to as guanosine monophosphate, reduced the overall HMG content, in particular Man 5 species, of the monoclonal antibodies produced. The discovery of such an effect of GMP on the HMG content of monoclonal antibodies expressed in CHO cells is surprising. Literature reports have indicated that feeding mannose can lead to higher levels of HMG, presumably due to higher concentrations of GDP-Mannose (Huang et al., Biotechnol. Bioeng., 2015, 112:1200-1209). One skilled in the art may expect that feeding GMP would have a similar impact, i.e., increasing the level of HMG. On the contrary, it was observed that feeding GMP reduced the level of HMG form, in particular Man 5, of the expressed proteins. Accordingly, an agent that increases GMP de novo synthesis, such as, but not limited to, Inosine 5′-monophosphate (IMP), Xanthine 5′-monophosphate (XMP), Guanosine 5′-diphosphate (GDP), Guanosine 5′-triphosphate (GTP), Guanosine 3′,5′-cyclic monophosphate (cGMP), Guanine, IMP dehydrogenase, or GMP synthase, can have similar effects on the HMG (e.g., Man 5) content of proteins (e.g., monoclonal antibodies) expressed during a mammalian cell culture (e.g., CHO cell culture) process.

These components can be supplemented either individually or in combination (e.g., of two, three, four, five, or more) to CHO cell cultures during a growth phase and/or a production phase to modify HMG levels in the expressed proteins. Such components can be used in batch or fed-batch cultures to modulate HMG levels, as well as in perfusion cultures where HMG levels may constantly need to be monitored as the culture progresses for a longer period. These components can also be used to maintain HMG levels in any mixed-generation processes, such as a combination of perfusion and fed-batch, or in expansion stages (accumulating cell biomass in series of passages, such as N-x, . . . , N-1) before a production stage. Because glycan species are usually not affected by the purification steps, modulating the expression level of various glycan species is critical for cell culture process.

The advantage of these components is to allow modification of HMG levels at will to refine and control final quality of product proteins expressed during a mammalian cell culture process. Furthermore, the supplementation of GMP has been shown to not negatively impact cell culture performance. These supplements can be used in commercial space to maintain consistent product quality and help reduce batch-to-batch variability in HMG levels by providing a blanket protection over lot-to-lot raw materials variability. As a result, these components can preserve and maintain the glycan quality of a therapeutic protein through its lifecycle in commercial space.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise. Similarly, the plural forms of words include their corresponding singular references unless the context clearly indicates otherwise.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values, and the ranges of the various combinations such as “from 50 mg to 100 mg”, “from 50 mg to 200 mg” and “from 100 mg to 300 mg”). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of” The terms “comprise(s),” “include(s),” “having,” “has,” “contain(s),” and equivalents thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds (or in the case of “consisting essentially of,” other components or compounds that materially impact the basic and novel properties of the claimed invention, i.e. the reduction of high mannose glycan content of a protein).

The term “protein” or “polypeptide” refers to a polymer of amino acid residues linked via sequential peptide bonds. In some embodiments, one or more amino acid residues can be an analog or mimetic of a naturally occurring amino acid. In certain embodiments, the “protein” or “polypeptide” comprises more than one polymer of amino acid residues, for example, an antibody that comprises two heavy chains and two light chains. The term can also encompass amino acid polymers that have been modified, e.g., by the addition of carbohydrate residues to form glycoproteins, or phosphorylated. Proteins comprise molecules having the amino acid sequence of a native protein, or molecules having deletions from, additions to, and/or substitutions of one or more amino acids of the native sequence.

The term “antibody,” “immunoglobulin,” or “Ig” is used interchangeably herein, and is used in the broadest sense and specifically encompasses, for example, individual monoclonal antibodies (including neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of the antibodies, as described below. An antibody can be human, humanized, chimeric and/or affinity matured, as well as an antibody from other species, for example, mouse and rabbit, etc. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments thereof) of any of the above, which refers to a portion of an antibody heavy or light chain polypeptide that retains some or all of the binding activity of the antibody from which the fragment was derived. Non-limiting examples of functional fragments (e.g., antigen-binding fragments thereof) include single-chain Fvs (scFv) (e.g., including monospecific, bispecific, etc.), Fab fragments, F(ab′) fragments, F(ab)₂ fragments, F(ab′)2 fragments, disulfide-linked Fvs (dsFv), Fd fragments, Fv fragments, diabody, triabody, tetrabody, and minibody. Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies can be of any class (e.g., IgG, IgE, IgM, IgD, and IgA) or any subclass (e.g., IgG1, IgG2, IgG3, IgG4, IgA1, and IgA2) of immunoglobulin molecule.

The term “monoclonal antibody” or “mAb” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J Mol. Biol. 222: 581-597, for example. See also Presta (2005) J Allergy Clin. Immunol. 116:731.

The terms “culture” and “cell culture” are used interchangeably and refer to a cell population that is maintained in a medium under conditions suitable to survival and/or growth of the cell population. As will be clear to those of ordinary skill in the art, these terms also refer to the combination comprising the cell population and the medium in which the population is maintained. Suitable culture conditions for mammalian cells are known in the art. See e.g. Animal cell culture: A Practical Approach, D. Rickwood, ed., Oxford University Press, New York (1992). Mammalian cells can be cultured in suspension or while attached to a solid substrate. Cell culture can be used to express a recombinant protein that is encoded by a gene of interest.

The term “cell culture medium,” “culture medium,” “nutrient feed medium,” “nutrient feed,” “culture feed,” “feed,” or “medium,” as used interchangeably herein, refers to any nutrient solution used for growing or maintaining cells (e.g., mammalian cells) and which generally provides at least one or more components from the following: essential and nonessential amino acids, vitamins, energy sources, lipids, and trace elements required by the cell for at least minimal growth and/or survival. The solution may also contain components that enhance growth and/or survival above the minimal rate, including hormones and growth factors. In at least one embodiment, the medium is a defined medium. Defined media are media in which all components have a known chemical structure. In other embodiments, the medium may contain an amino acid(s) derived from any source or method known in the art, including, but not limited to, an amino acid(s) derived either from single amino acid addition(s) or from a peptone or protein hydrolysate addition(s) (including animal or plant source(s)). Cell culture medium includes those that are typically employed in and/or are known for use with any cell culture process, including but not limited to, batch, extended batch, fed-batch, perfusion or continuous culturing of cells.

The term “essentially same” as used in connection with “cell culture” means that the cell culture conditions and the components and amounts of the rest of the cell culture media are the same except for the presence or absence of GMP or an agent that increases GMP de novo synthesis described herein. The term “essentially same” as used in connection with “cell culture medium” means that the components and amounts of the rest of the media are the same except for the presence or absence of GMP or an agent that increases GMP de novo synthesis described herein.

When used in connection with a cell culture herein, “growth phase” refers to the period from inoculation of the cell culture to the time when viable cell density reaches a plateau, including but not limited to an exponential growth or log period; “production phase” refers to the period when cell proliferation slows or suspends and the cells use energy and substrates to produce a protein of interest instead of making more cells. A transition from “growth phase” to “production phase” can be achieved by various means known by a skilled person in the art, including but not limited to, temperature shift, pH shift, amino acid starvation, or use of a cell-cycle inhibitor or other molecule that can arrest cell growth without causing cell death.

“High mannose glycan” or “HMG” refers to an N-glycosylation form of a protein that contains unsubstituted terminal mannose sugars. These glycans typically contain between five and nine mannose residues attached to the chitobiose (GlcNAc₂) core. The name abbreviations are indicative of the total number of mannose residues in the structure. In one embodiment, the high mannose glycan species is Mannose 5 (Man 5). In another embodiment, the high mannose glycan species is Mannose 6 (Man 6), Mannose 7 (Man 7), Mannose 8 (including Mannose 8a (Man 8a) and 8b (Man 8b)), or Mannose 9 (Man 9). In a further embodiment, the high mannose glycan species comprise a mixture of Man 5, Man 6, Man 7, Man 8a, Man 8b, and/or Man 9. The HMG content of a protein can be measured using any methods known in the art, including but not limited to ultra-performance liquid chromatography (UPLC) with 2-aminobenzamide (2-AB) labeling as described herein.

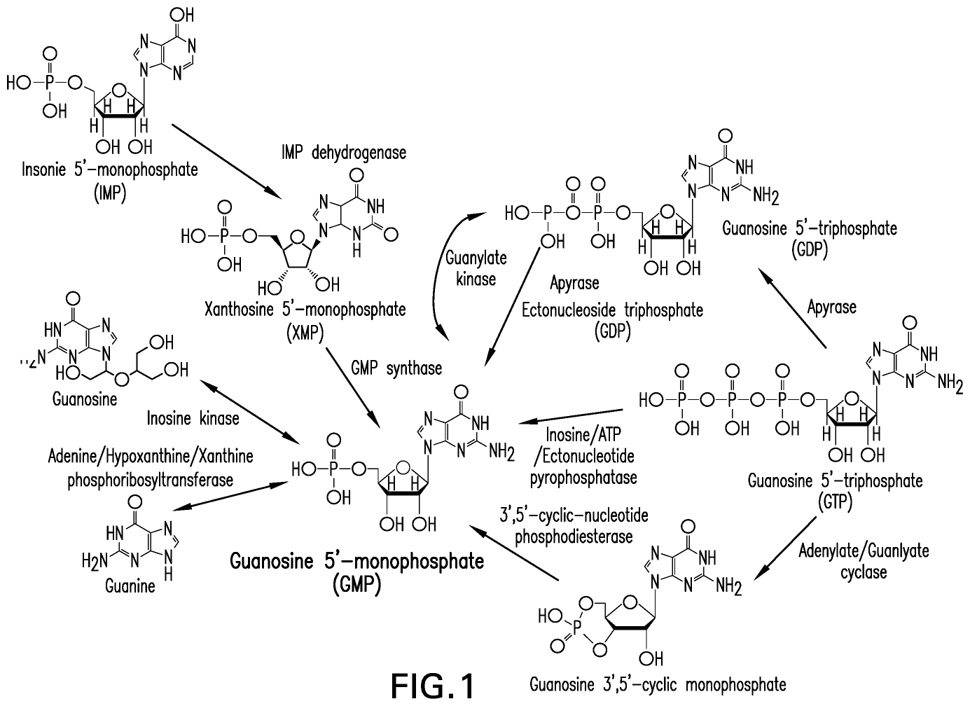

The term “GMP de novo synthesis” as used herein refers to the synthesis of Guanosine 5′-monophosphate by cells. The exemplary pathways of GMP de novo synthesis and the chemical structures for various components are shown in FIG. 1. The most common pathway to GMP synthesis de novo is formation of GMP from XMP, which is in turn synthesized from IMP, the reactions catalyzed by GMP synthase (Hirst et al., J Biol Chem, 1994, 269:23830-7) and IMP dehydrogenase (Stee et al., Mol. Gen. Genet., 1995, 248:755-766), respectively. GMP can also be synthesized from cGMP as cGMP will convert to GMP in the presence of phosphodiesterases (Nikawa et al., Mol. Cell. Biol., October 1987, p. 3629-3636). GMP can also be synthesized by either GTP directly using pyrophosphatase enzymes (Lin et al., J. Biol. Chem. 2001, 276:18695-18701; Belli and Goding, Eur. J. Biochem., 1994, 226:433-443; Johnson et al., Biochem., 54:625-629) or GTP converting to GDP and then further to GMP by either using reversible guanylate kinase (Beck et al., Journal of Bacteriology, 2003, 185(22):6732-6735) or apyrase and diphosphohydrylase (Maliszewski et al., J Immunol, 1994, 153:3574-83; Chadwick et al., Genomics, 1998, 50(3):357-367). Furthermore, guanine can either directly convert to GMP using phophoribosyltransferases (Guddat et al., Protein Science, 2002, 11:1626-1638; Vos et al., Biochemistry, 1997, 36(14):4125-34) or convert to guanosine that can become phosphorylated to become GMP (Harlow et al., J. Bacteriology, 1995, 177(8):2236-2240).

The term “batch” or “batch culture” as used herein refers to a method of culturing cells in which all the components that will ultimately be used in culturing the cells, including the medium as well as the cells themselves, are provided at the beginning of the culturing process. A batch culture is typically stopped at some point and the cells and/or components in the medium are harvested and optionally purified.

The term “fed batch” or “fed-batch” as used herein refers to a method of culturing cells in which additional components are provided to the culture at some time subsequent to the beginning of the culture process. The provided components typically comprise nutritional supplements for the cells that have been depleted during the culturing process. A fed-batch culture is typically stopped at some point and the cells and/or components in the medium are harvested and optionally purified.

The term “perfusion” as used herein refers to a method of culturing cells in which additional fresh medium is provided, either continuously over some period of time or intermittently over some period of time, to the culture (subsequent to the beginning of the culture process), and simultaneously spent medium is removed. The fresh medium typically provides nutritional supplements for the cells that have been depleted during the culturing process. Polypeptide product, which may be present in the spent medium, is optionally purified. Perfusion also allows for removal of cellular waste products from the cell culture.

The term “viability” means the ability of cells in culture to survive under a given set of culture conditions or experimental variations. The term also refers to that portion of cells which are alive at a particular time in relation to the total number of cells, living and dead, in the culture at that time.

“Viable cell density” or “VCD” refers to the number of live cells in a given volume of culture medium, as determined by standard viability assays (such as trypan blue dye exclusion method).

The terms “integrated viable cell density”, “integral viable cell concentration”, “IVCC”, or “IVCD” are used interchangeably and mean the average density of viable cells over the course of the culture multiplied by the amount of time the culture has run.

The term “titer” means the total amount of a polypeptide or protein produced by a cell culture in a given amount of medium volume. Titer can be expressed in units of milligrams or micrograms of polypeptide or protein per milliliter (or other measure of volume) of medium. “Cumulative titer” is the titer produced by the cells during the course of the culture, and can be determined, for example, by measuring daily titers and using those values to calculate the cumulative titer.

“Final concentration,” when used in connection with GMP or an agent that increases GMP de novo synthesis, refers to the concentration of GMP or the agent that increases GMP de novo synthesis in a cell culture wherein the cells are in contact with GMP or the agent that increases GMP de novo synthesis. “Final concentration” of GMP or an agent that increases GMP de novo synthesis in a “cell culture medium” refers to the concentration of GMP or the agent that increases GMP de novo synthesis in a cell culture medium wherein GMP or the agent that increases GMP de novo synthesis is in contact with the cells. Unless indicated otherwise (e.g., in a stock solution), the concentrations of GMP described herein are final concentrations of GMP in a cell culture or a cell culture medium.

Methods of Reducing a HMG content of a Protein Expressed During a Mammalian Cell Culture Process

In one aspect, the present disclosure provides a method for reducing an HMG content of a protein (e.g., monoclonal antibody) expressed during a mammalian cell culture (e.g., CHO cell culture) process.

In some embodiments, provided is a method of reducing an HMG content of a protein expressed during a mammalian cell culture process, comprising:
(1) establishing a mammalian cell culture expressing the protein; and
(2) contacting the cell culture with Guanosine 5′-monophosphate (GMP) or an agent that increases GMP de novo synthesis.

In one embodiment, the mammalian cell culture is established by inoculating a cell culture container with at least 0.5×10⁶-3×10⁶ cells/mL in a cell culture medium. In another embodiment, the mammalian cell culture is established by inoculating a cell culture container with at least 0.5×10⁶-1.5×10⁶ cells/mL in a cell culture medium. In yet another embodiment, the mammalian cell culture is established by inoculating a cell culture container with at least 1×10⁶-2×10⁶ cells/mL in a cell culture medium. In still another embodiment, the mammalian cell culture is established by inoculating a cell culture container with at least 0.5×10⁶-1×10⁶ cells/mL in a cell culture medium. In one embodiment, the mammalian cell culture is established by inoculating a cell culture container with about 0.5×10⁶ cells/mL in a cell culture medium. In another embodiment, the mammalian cell culture is established by inoculating a cell culture container with about 1×10⁶ cells/mL in a cell culture medium. In one embodiment, the mammalian cell culture is established by inoculating a cell culture container with about 2×10⁶ cells/mL in a cell culture medium. In another embodiment, the mammalian cell culture is established by inoculating a cell culture container with about 3×10⁶ cells/mL in a cell culture medium. In one embodiment, the mammalian cell culture is established by inoculating a cell culture container with more than 3×10⁶ cells/mL in a cell culture medium. In another embodiment, the mammalian cell culture is established by inoculating a cell culture container with more than 4×10⁶ cells/mL in a cell culture medium.

In certain embodiments of the method, step (2) occurs during a growth phase of the cell culture.

In some embodiments of the method, step (2) occurs during a production phase of the cell culture.

In other embodiments of the method, step (2) occurs during a growth phase and a production phase of the cell culture.

In some embodiments of various methods described herein, step (2) comprises contacting the cell culture with GMP.

In other embodiments of various methods described herein, step (2) comprises contacting the cell culture with an agent that increases GMP de novo synthesis.

In various embodiments, the agent is Inosine 5′-monophosphate (IMP), Xanthine 5′-monophosphate (XMP), Guanosine 5′-diphosphate (GDP), Guanosine 5′-triphosphate (GTP), Guanosine 3′, 5′-cyclic monophosphate (cGMP), Guanine, IMP dehydrogenase, GMP synthase, or a combination thereof. In specific embodiments, the agent is Inosine 5′-monophosphate (IMP), Xanthine 5′-monophosphate (XMP), Guanosine 5′-diphosphate (GDP), Guanosine 5′-triphosphate (GTP), Guanosine 3′, 5′-cyclic monophosphate (cGMP), Guanine, IMP dehydrogenase, or GMP synthase. In one embodiment, the agent is IMP. In another embodiment, the agent is XMP. In yet another embodiment, the agent is GDP. In still another embodiment, the agent is GTP. In yet still another embodiment, the agent is cGMP. In one embodiment, the agent is Guanine. In another embodiment, the agent is IMP dehydrogenase. In yet another embodiment, the agent is GMP synthase.

Thus, in one embodiment, provided is a method of reducing a HMG content of a protein expressed during a mammalian cell culture process, comprising:
(1) establishing a mammalian cell culture expressing the protein; and
(2) contacting the cell culture with GMP during a growth phase.

In another embodiment, provided is a method of reducing a HMG content of a protein expressed during a mammalian cell culture process, comprising:
(1) establishing a mammalian cell culture expressing the protein; and
(2) contacting the cell culture with GMP during a production phase.

In yet another embodiment, provided is a method of reducing a HMG content of a protein expressed during a mammalian cell culture process, comprising:
(1) establishing a mammalian cell culture expressing the protein; and
(2) contacting the cell culture with GMP during a growth phase and a production phase.

In certain embodiments of the various methods described herein, the GMP is present in the cell culture for a time period of 1, 2, 3, 4, 5, 6, 7, 8, 9, or 10 days or longer. In one embodiment, the GMP is present in the cell culture for 1 day. In another embodiment, the GMP is present in the cell culture for 2 days. In yet another embodiment, the GMP is present in the cell culture for 3 days. In still another embodiment, the GMP is present in the cell culture for 4 days. In yet still another embodiment, the GMP is present in the cell culture for 5 days. In one embodiment, the GMP is present in the cell culture for 6 days. In another embodiment, the GMP is present in the cell culture for 7 days. In yet another embodiment, the GMP is present in the cell culture for 8 days. In still another embodiment, the GMP is present in the cell culture for 9 days. In yet still another embodiment, the GMP is present in the cell culture for 10 days or longer.

In other embodiments, the GMP is present in the cell culture for the entire duration of the cell culture.

In some embodiments, the final concentration of GMP in the cell culture is from 1 mM to 50 mM, from 1 mM to 40 mM, from 1 mM to 30 mM, for 1 mM to 25 mM, or from 1 mM to 20 mM. In certain embodiments, the final concentration of GMP in the cell culture is from 1 mM to 20 mM, from 3 mM to 20 mM, from 3 mM to 18 mM, from 3 mM to 15 mM, from 5 mM to 15 mM, or from 3 mM to 10 mM. In other embodiments, the final concentration of GMP in the cell culture is 1 mM, 3 mM, 5 mM, 10 mM, 15 mM, 18 mM, 20 mM, or 25 mM. In one embodiment, the final concentration of GMP in the cell culture is 1 mM. In another embodiment, the final concentration of GMP in the cell culture is 3 mM. In yet another embodiment, the final concentration of GMP in the cell culture is 5 mM. In still another embodiment, the final concentration of GMP in the cell culture is 10 mM. In one embodiment, the final concentration of GMP in the cell culture is 15 mM. In another embodiment, the final concentration of GMP in the cell culture is 18 mM. In yet another embodiment, the final concentration of GMP in the cell culture is 20 mM. In still another embodiment, the final concentration of GMP in the cell culture is 25 mM.

In one embodiment, provided is a method of reducing a HMG content of a protein expressed during a mammalian cell culture process, comprising:
(1) establishing a mammalian cell culture expressing the protein; and
(2) contacting the cell culture with 1-20 mM GMP during a growth phase.

In another embodiment, provided is a method of reducing a HMG content of a protein expressed during a mammalian cell culture process, comprising:
(1) establishing a mammalian cell culture expressing the protein; and
(2) contacting the cell culture with 1-20 mM GMP during a production phase.

In yet another embodiment, provided is a method of reducing a HMG content of a protein expressed during a mammalian cell culture process, comprising:
(1) establishing a mammalian cell culture expressing the protein; and
(2) contacting the cell culture with 1-20 mM GMP during a growth phase and a production phase.

In some embodiments, the GMP is added to the cell culture 1-15 days, 3-15 days, or 5-15 days after the cell culture is established. In certain embodiments, the GMP is added to the cell culture at day 3, at day 4, at day 5, at day 6, at day 7, at day 8, at day 9, at day 10, at day 11, at day 12, at day 13, at day 14, or at day 15 after the cell culture is established. In one embodiment, the GMP is added to the cell culture at day 3 after the cell culture is established. In another embodiment, the GMP is added to the cell culture at day 4 after the cell culture is established. In yet another embodiment, the GMP is added to the cell culture at day 5 after the cell culture is established. In still another embodiment, the GMP is added to the cell culture at day 6 after the cell culture is established. In one embodiment, the GMP is added to the cell culture at day 7 after the cell culture is established. In another embodiment, the GMP is added to the cell culture at day 8 after the cell culture is established. In yet another embodiment, the GMP is added to the cell culture at day 9 after the cell culture is established. In still another embodiment, the GMP is added to the cell culture at day 10 after the cell culture is established. In one embodiment, the GMP is added to the cell culture at day 11 after the cell culture is established. In another embodiment, the GMP is added to the cell culture at day 12 after the cell culture is established. In yet another embodiment, the GMP is added to the cell culture at day 13 after the cell culture is established. In still another embodiment, the GMP is added to the cell culture at day 14 after the cell culture is established. In yet still another embodiment, the GMP is added to the cell culture at day 15 after the cell culture is established.

In certain embodiments, the GMP is added to the cell culture 1-15 days, 3-15 days, or 5-15 days before the cell culture is harvested. In certain embodiments, the GMP is added to the cell culture 1, 2, 3, 4, 5, 6, 7, 8, 9, or 10 days before the cell culture is harvested. In one embodiment, the GMP is added to the cell culture 1 day before the cell culture is harvested. In another embodiment, the GMP is added to the cell culture 2 days before the cell culture is harvested. In yet another embodiment, the GMP is added to the cell culture 3 days before the cell culture is harvested. In still another embodiment, the GMP is added to the cell culture 4 days before the cell culture is harvested. In one embodiment, the GMP is added to the cell culture 5 days before the cell culture is harvested. In another embodiment, the GMP is added to the cell culture 6 days before the cell culture is harvested. In yet another embodiment, the GMP is added to the cell culture 7 days before the cell culture is harvested. In still another embodiment, the GMP is added to the cell culture 8 days before the cell culture is harvested. In one embodiment, the GMP is added to the cell culture 9 days before the cell culture is harvested. In another embodiment, the GMP is added to the cell culture 10 days before the cell culture is harvested.

In other embodiments, the GMP is added to the cell culture when viable cell density (VCD) is at least about 0.5×10⁶-1.5×10⁶, 0.5×10⁶-3×10⁶, 1×10⁶-3×10⁶, 3×10⁶-5×10⁶, 5×10⁶-10×10⁶, 10×10⁶-20×10⁶, 20×10⁶-30×10⁶, 30×10⁶-40×10⁶, 40×10⁶-50×10⁶, 50×10⁶-60×10⁶, 60×10⁶-70×10⁶, 70×10⁶-80×10⁶, 80×10⁶-90×10⁶, or 90×10⁶-100×10⁶ viable cells/mL. In certain embodiments, the GMP is added to the cell culture when the VCD is at least about 10×10⁶-80×10⁶, 10×10⁶-70×10⁶, 10×10⁶-60×10⁶, 10×10⁶-50×10⁶, 10×10⁶-40×10⁶, 10×10⁶-30×10⁶, 10×10⁶-15×10⁶, 15×10⁶-20×10⁶, 20×10⁶-25×10⁶, 25×10⁶-30×10⁶, 30×10⁶-35×10⁶, or 35×10⁶-40×10⁶ viable cells/mL. In one embodiment, the viable cell density is at least about 10×10⁶ viable cells/mL to 80×10⁶ viable cells/mL. In another embodiment, the viable cell density is at least about 10×10⁶ viable cells/mL to 70×10⁶ viable cells/mL. In yet another embodiment, the viable cell density is at least about 10×10⁶ viable cells/mL to 60×10⁶ viable cells/mL. In still another embodiment, the viable cell density is at least about 10×10⁶ viable cells/mL to 50×10⁶ viable cells/mL. In one embodiment, the viable cell density is at least about 10×10⁶ viable cells/mL to 40×10⁶ viable cells/mL. In another embodiment, the viable cell density is at least about 10×10⁶ viable cells/mL to 30×10⁶ viable cells/mL. In yet another embodiment, the viable cell density is at least about 10×10⁶ viable cells/mL to 20×10⁶ viable cells/mL. In still another embodiment, the viable cell density is at least about 20×10⁶ viable cells/mL to 30×10⁶ viable cells/mL. In still another embodiment, the viable cell density is at least about 30×10⁶ viable cells/mL to 50×10⁶ viable cells/mL. In still another embodiment, the viable cell density is at least about 30×10⁶ viable cells/mL to 60×10⁶ viable cells/mL. In yet still another embodiment, the viable cell density is at least about 20×10⁶ viable cells/mL to at least about 25×10⁶ viable cells/mL, or at least about 20×10⁶ viable cells/mL. In yet still another embodiment, the viable cell density is at least about 30×10⁶ viable cells/mL to at least about 50×10⁶ viable cells/mL, or at least about 40×10⁶ viable cells/mL.

In one embodiment, the GMP is included in the medium, which can be a feed medium or a perfusion medium, at a selected final concentration (e.g., 1 mM, 3 mM, 5 mM, 10 mM, 18 mM, or 20 mM). In another embodiment, the GMP is added to the cell culture along with the medium. In yet another embodiment, the GMP is added to the cell culture separately from the medium. The GMP is added to the cell culture at a rate sufficient to achieve and/or maintain a desired final concentration in the cell culture. In one embodiment, the GMP is added at a rate of 1/40- 1/60 of the rate at which the medium is added to the cell culture, for example, by perfusion. In another embodiment, the GMP is added at a rate of 1/50 of the rate at which the medium is added to the cell culture, for example, by perfusion. In further embodiments, the rate of adding GMP is varied to achieve a desired final concentration using calculations that are known in the art. The GMP can be added at a rate that is from 1/10 of that of the culture medium to 1/100 of that of the culture medium.

In certain embodiments of various methods described herein, the cell culture is maintained by batch culture for a partial or entire period of the cell culture. In one embodiment, the batch begins on day 1 of the cell culture. In another embodiment, the batch begins when the cell culture reaches a production phase.

In certain embodiments of various methods described herein, the cell culture is maintained by fed batch for a partial or entire period of the cell culture. In one embodiment, the fed batch begins on day 1 of the cell culture. In another embodiment, the fed batch begins when the cell culture reaches a production phase. In some embodiments, the feed medium is added to the cell culture every day, every 2 days, every 3 days, every 4 days, or every 5 days, throughout the entire period of the cell culture or during the production phase. In one embodiment, the culture is fed three times during the production phase. In another embodiment, the culture is fed four times during the production phase. In yet another embodiment, the culture is fed on a day between day two and four, on a day between day 5 and 7, and on a day between day 8 and 10 of the production phase. In yet another embodiment, the culture is fed on a day between day two and four, on a day between day 5 and 6, on a day between day 7 and 8, and on a day between day 8 and 10 or later of the production phase.

In one embodiment, GMP is added to a fed batch culture on the days when the feed medium is added to the culture. For example, GMP can be added three or four times during the production phase, at the times set forth previously. GMP can be added directly to the cell culture (separately from the feed medium) at a concentration designed to achieve a specific final concentration in the culture. Or GMP can be added to the feed medium first at a concentration designed to achieve a specific final concentration in the culture, then the feed medium is added to the cell culture. In another embodiment, GMP is added directly to the culture on a day or days when the culture is not being fed (i.e., no additional feed medium is added).

In some embodiments of various methods described herein, the cell culture is maintained by perfusion for a partial or entire period of the cell culture. In one embodiment, the perfusion begins on or about day 1 to on or about day 9 of the cell culture. In another embodiment, the perfusion begins on or about day 3 to on or about day 7 of the cell culture. In yet another embodiment, the perfusion begins on day 1 of the cell culture. In still another embodiment, the perfusion begins when the cell culture reaches a production phase. In some embodiments, perfusion is accomplished by alternating tangential flow. In other embodiments, the perfusion is accomplished by alternating tangential flow using an ultrafilter or a microfilter.

Perfusion can be continuous, stepwise, intermittent, or a combination of any or all of these. In certain embodiments, the rate of perfusion is constant. In some embodiments, the rate of perfusion varies. In one embodiment, the perfusion is performed at a rate of less than or equal to 1.0 working volumes per day. In another embodiment, the perfusion is performed at a rate that increases during the production phase from 0.25 working volume per day to 1.0 working volume per day. In yet another embodiment, the perfusion is performed at a rate that reaches 1.0 working volume per day on day 9 to day 11 of the cell culture. In still another embodiment, the perfusion is performed at a rate that reaches 1.0 working volume per day on day 10 of the cell culture.

In other embodiments of various methods described herein, the cell culture is maintained by a combination of batch and perfusion or a combination of fed batch and perfusion for a partial or entire period of the cell culture. In some embodiments, the cell culture is maintained by batch or fed batch in the growth phase and by perfusion in the production phase. In one embodiment, perfusion begins on or about day 2 to on or about day 9 of the cell culture. In another embodiment, perfusion begins on or about day 2 to on or about day 7 of the cell culture. In yet another embodiment, perfusion begins on or about day 3 to on or about day 9 of the cell culture. In still another embodiment, perfusion begins on or about day 3 to on or about day 7 of the cell culture. In yet still another embodiment, perfusion begins on or about day 5 to on or about day 7 of the cell culture. In one embodiment, perfusion begins before the cell culture reaches the production phase. In another embodiment, perfusion begins at or about the time when the cell culture reaches the production phase. In yet another embodiment, perfusion begins after the cell culture reaches the production phase.

In some embodiments, such as multiple phase cell culture process, cells are cultured in two or more distinct phases. For example, cells can be cultured first in one or more growth phases, under environmental conditions that maximize cell proliferation and viability, then transferred to a production phase, under conditions that maximize protein production. In a commercial process for production of a protein by mammalian cells, there are commonly multiple, for example, at least about 2, 3, 4, 5, 6, 7, 8, 9, or 10 growth phases (e.g., N-1, N-2, etc.) that occur in different culture vessels preceding a final production stage. In some embodiments, the final production stage comprises a growth phase and a production phase.

In other embodiments, the growth and production phases can be preceded by, or separated by, one or more transition phases. In multiple phase processes, the method described herein can be employed during the growth and production phase of the final production stage of a commercial cell culture, or in a preceding transition phase or growth phase (e.g., N-1, N-2, etc.). A production phase can be conducted at large scale. A large-scale process can be conducted in a volume of at least about 100, 500, 1000, 2000, 3000, 5000, 7000, 8000, 10,000, 15,000, or 20,000 liters. In some embodiments, production is conducted in 500 L, 1000 L, or 2000 L bioreactors.

In some embodiments, the HMG content of the protein is reduced by at least 1%, 3%, 5%, 10%, 15%, 20%, 25%, or 30% compared to the HMG content of the protein expressed in an essentially same cell culture except that the essentially same cell culture is not contacted with GMP or the agent that increases GMP de novo synthesis. In one embodiment, the HMG content of the protein is reduced by at least 3%. In another embodiment, the HMG content of the protein is reduced by at least 5%. In yet another embodiment, the HMG content of the protein is reduced by at least 10%. In still another embodiment, the HMG content of the protein is reduced by at least 20%. In yet still another embodiment, the HMG content of the protein is reduced by at least 30%.

In one embodiment, provided is a method of reducing a HMG content of a protein expressed during a mammalian cell culture process, comprising:
(1) establishing a mammalian cell culture expressing the protein; and
(2) contacting the cell culture with 1-20 mM GMP during a growth phase;
   wherein the HMG content of the protein is reduced by at least 3%, 5%, 10%, 20%, or 30% compared to the HMG content of the protein expressed in an essentially same cell culture except that the essentially same cell culture is not contacted with GMP.

In another embodiment, provided is a method of reducing a HMG content of a protein expressed during a mammalian cell culture process, comprising:
(1) establishing a mammalian cell culture expressing the protein; and
(2) contacting the cell culture with 1-20 mM GMP during a production phase;
   wherein the HMG content of the protein is reduced by at least 3%, 5%, 10%, 20%, or 30% compared to the HMG content of the protein expressed in an essentially same cell culture except that the essentially same cell culture is not contacted with GMP.

In yet another embodiment, provided is a method of reducing a HMG content of a protein expressed during a mammalian cell culture process, comprising:
(1) establishing a mammalian cell culture expressing the protein; and
(2) contacting the cell culture with 1-20 mM GMP during a growth phase and a production phase;
   wherein the HMG content of the protein is reduced by 3%, 5%, 10%, 20%, or 30% compared to the HMG content of the protein expressed in an essentially same cell culture except that the essentially same cell culture is not contacted with GMP.

In certain embodiments, the HMG is Man 5. In some embodiments, the HMG is Man 6, Man 7, Man 8a, Man 8b, or Man 9. In other embodiments, the HMG is a mixture of one or more species selected from the group consisting of Man 5, Man 6, Man 7, Man 8a, Man 8b, and Man 9.

In some embodiments of various methods described herein, the method further comprises a harvest step.

In another embodiment, the protein produced by the cell culture is purified and formulated in a pharmaceutically acceptable formulation.

A wide variety of mammalian cell lines suitable for growth in culture are available from the American Type Culture Collection (Manassas, Va.) and commercial vendors. Examples of cell lines commonly used include VERO, BHK, HeLa, CV1 (including Cos), MDCK, 293, 3T3, myeloma cell lines (e.g., NSO, NS1), PC12, W138 cells, and Chinese hamster ovary (CHO) cells. In certain embodiments, the mammalian cell culture is a CHO cell culture. Suitable CHO cell lines include but are not limited to CHO, CHO-K1, CHO-DUKX, CHO-DUKX B1, CHO-DG44, CHO-DBX11, CHOK1SV™, HD-BIOP1, CHOZN®, BHK21, BHK TK, or ExpiCHO, as well as derivatives/descendants of these CHO cell lines.

Non-limiting examples of proteins that can be produced in the disclosed methods include therapeutic proteins, monoclonal antibodies, hormones, cytokines, growth factors, clotting factors, enzymes, fusion proteins thereof, immunoconjugates thereof, and fragments thereof. In certain embodiments, the protein is a therapeutic protein. In some embodiments, the protein is a monoclonal antibody. In other embodiments, the protein is a hormone. In yet other embodiments, the protein is a cytokine. In still other embodiments, the protein is a growth factor. In certain embodiments, the protein is a clotting factor. In some embodiments, the protein is an enzyme. In other embodiments, the protein is a fusion protein of the above proteins. In yet other embodiments, the protein is an immunoconjugate of the above proteins. In still other embodiments, the protein is a fragment of the above proteins.

### Cell Culture Media for Reducing a HMG Content of a Protein Expressed During a Mammalian Cell Culture Process

In another aspect, provided is a cell culture medium for reducing a HMG content of a protein (e.g., monoclonal antibody) expressed during a mammalian cell culture (e.g., CHO cell culture) process, comprising GMP or an agent that increases GMP de novo synthesis.

In certain embodiments of various media described herein, the GMP or the agent that increases GMP de novo synthesis is at a final concentration that is sufficient to reduce the HMG content of the protein by at least 1%, 3%, 5%, 10%, 15%, 20%, 25%, or 30% compared to the protein expressed in an essentially same cell culture medium except that the essentially same cell culture medium does not include GMP or the agent that increases GMP de novo synthesis.

In one embodiment, the cell culture medium comprises GMP. In some embodiments, the final concentration of GMP in the cell culture is from 1 mM to 50 mM, from 1 mM to 40 mM, from 1 mM to 30 mM, for 1 mM to 25 mM, or from 1 mM to 20 mM. In certain embodiments, the final concentration of GMP in the cell culture is from 1 mM to 20 mM, from 3 mM to 20 mM, from 3 mM to 18 mM, from 3 mM to 15 mM, from 5 mM to 15 mM, or from 3 mM to 10 mM. In other embodiments, the final concentration of GMP in the cell culture is 1 mM, 3 mM, 5 mM, 10 mM, 15 mM, 18 mM, 20 mM, or 25 mM. In one embodiment, the final concentration of GMP in the cell culture is 1 mM. In another embodiment, the final concentration of GMP in the cell culture is 3 mM. In yet another embodiment, the final concentration of GMP in the cell culture is 5 mM. In still another embodiment, the final concentration of GMP in the cell culture is 10 mM. In one embodiment, the final concentration of GMP in the cell culture is 15 mM. In another embodiment, the final concentration of GMP in the cell culture is 18 mM. In yet another embodiment, the final concentration of GMP in the cell culture is 20 mM. In still another embodiment, the final concentration of GMP in the cell culture is 25 mM.

In one embodiment, provided is a cell culture medium for reducing a HMG content of a protein expressed during a mammalian cell culture process comprising GMP, wherein the GMP final concentration in the cell culture is 1-20 mM.

In another embodiment, provided is a cell culture medium for reducing a HMG content of a protein expressed during a mammalian cell culture process comprising GMP, wherein the GMP final concentration in the cell culture is sufficient to reduce the HMG content of the protein by at least 1%, 3%, 5%, 10%, 15%, 20%, 25%, or 30% compared to the protein expressed in an essentially same cell culture medium except that the essentially same cell culture medium does not include GMP.

In yet another embodiment, provided is a cell culture medium for reducing a HMG content of a protein expressed during a mammalian cell culture process comprising GMP, wherein the GMP final concentration in the cell culture is 1-20 mM and sufficient to reduce the HMG content of the protein by at least 1%, 3%, 5%, 10%, 15%, 20%, 25%, or 30% compared to the protein expressed in an essentially same cell culture medium except that the essentially same cell culture medium does not include GMP.

In another embodiment, the cell culture medium comprises an agent that increases GMP de novo synthesis. In various embodiments, the agent is Inosine 5′-monophosphate (IMP), Xanthine 5′-monophosphate (XMP), Guanosine 5′-diphosphate (GDP), Guanosine 5′-triphosphate (GTP), Guanosine 3′, 5′-cyclic monophosphate (cGMP), Guanine, IMP dehydrogenase, or GMP synthase. In one embodiment, the agent is IMP. In another embodiment, the agent is XMP. In yet another embodiment, the agent is GDP. In still another embodiment, the agent is GTP. In yet still another embodiment, the agent is cGMP. In one embodiment, the agent is Guanine. In another embodiment, the agent is IMP dehydrogenase. In yet another embodiment, the agent is GMP synthase.

In some embodiments, the cell culture medium is a mammalian cell culture medium. In other embodiments, the cell culture medium is a CHO cell culture medium.

The cell culture medium described herein can be used at any stage of a cell culture. For example, the cell culture medium can be used as a base medium, a growth medium, a production medium, a batch medium, a feed medium, or a perfusion medium.

The cell culture medium can be supplemented with any additional optional components known in the art to optimize growth of cells and/or expression of proteins, such as hormones and other growth factors, e.g., insulin, transferrin, epidermal growth factor, serum, and the like; salts, e.g., calcium, magnesium and phosphate; buffers, e.g., HEPES; nucleosides and bases, e.g., adenosine, thymidine, hypoxanthine; protein and tissue hydrolysates, e.g., hydrolyzed animal or plant protein; antibiotics, e.g., gentamycin; cell protectants or surfactants, e.g., Pluronic F-68; or polyamines, e.g., putrescine, spermidine and spermine, depending on the requirements of the cells to be cultured and/or the desired cell culture parameters.

Further, the cell culture medium can be concentrated cell culture medium that contains some or all of the nutrients necessary to maintain the cell culture. In particular, concentrated medium can contain nutrients identified as or known to be consumed during the course of the production phase of the cell culture. Concentrated medium can be based on any cell culture media formulation. Such a concentrated feed medium can contain some or all the components of the cell culture medium at, for example, about 2×, 3×, 4×, 5×, 6×, 7×, 8×, 9×, 10×, 12×, 14×, 16×, 20×, 30×, 50×, 100×, 200×, 400×, 600×, 800×, or even about 1000× of their normal amount.

### EXAMPLES

### Example 1. Effect of Various Components on HMG Content of Proteins and Cell Culture Parameters

The effect of eighteen unique components, including GMP, various sugars, nucleotide precursors, glycosylation pathway substrates, or mineral salts, on recombinant CHO cell lines expressing monoclonal antibodies (mAb A, mAb B, or mAb C) that exhibit low, medium, or high levels of HMG content, respectively, was assessed in a 10-day fed-batch assay. Experiment was executed by dividing into 6-blocks. In each experiment block, 3 unique test components were assessed and control conditions with no added test component were included for all three cell lines. Cells were cultured in Merck proprietary chemically defined media in increasing volume to obtain sufficient number of cells for inoculation. For cell lines expressing mAb A and mAb B, cells were seeded at 0.5×10⁶ cells per mL into Merck proprietary chemically defined production medium in two 1 L Erlenmeyer flasks at a final volume of 250 mL in each flask. For cell line expressing mAb C, cells were seeded at 1.0×10⁶ cells per mL into production medium in two 1 L Erlenmeyer flasks at a final volume of 250 mL in each flask. Cells were incubated at 36.5° C., with agitation at 100 rpm, in presence of 5% CO2, and humidity ≥70%. Merck proprietary chemically defined nutrient feed media were added to all the cultures on day 3, day 6, and day 8. On Day 6, cells from both 1 L flasks for each cell line were pooled and then aliquoted in 125 mL Erlenmeyer flasks with 30 mL working volume, respectively. Stock solutions for each of the tested component were prepared as defined in Table 1 below, and all components were ordered from Sigma Aldrich (St. Louis, MO). Component 6 is GMP. All solutions were prepared in purified water. For each cell line, components were added to the cultures at final concentration at a lower and higher concentrations as per Table 1, respectively, on day 6 of the fed-batch culture. No significant changes in pH and osmolality were observed as a result of the components additions. A control condition with no added test component was also created for each cell line. All conditions were run with two replicates. All cultures were harvested on day 10.

TABLE 1
Eighteen components tested for cell
lines producing mAb A, mAb B or mAB C

| | Lower | Higher | Stock |
|---|---|---|---|
| | Concentration | Concentration | solution |
| | Tested | Tested | Concentration |
| Component | (mM) | (mM) | (mM) |
| 1 | 1 | 10 | 400 |
| 2 | 1 | 10 | 400 |
| 3 | 5 | 20 | 500 |
| 4 | 5 | 20 | 800 |
| 5 | 0.01 | 0.1 | 10 |
| 6 - GMP | 1 | 10 | 200 |
| 7 | 1 | 10 | 400 |
| 8 | 1 | 10 | 400 |
| 9 | 1 | 10 | 400 |
| 10 | 0.1 | 0.5 | 200 |
| 11 | 0.1 | 0.5 | 20 |
| 12 | 1 | 10 | 400 |
| 13 | 10 | 50 | 2000 |
| 14 | 0.002 | 0.02 | 1 |
| 15 | 0.002 | 0.02 | 1 |
| 16 | 5 | 20 | 800 |
| 17 | 5 | 20 | 1000 |
| 18 | 0.1 | 0.5 | 20 |

Cell culture parameters were analyzed on day 0, 3, 6, 8 and 10, and the corresponding spent medium supernatants were evaluated for antibody titer, charge variants and glycan analysis on day 10 to assess the effect of added components on cell growth, viability, titer, aggregation, charge variants and glycan profile of the recombinant antibodies. Cell density and viability were measured using the trypan blue exclusion method with Cedex Bio Analyzer (Roche Diagnostics GmbH, Mannheim, Germany). Culture pH was measured using ABL80 Blood/Gas Analyzer (Radiometer, Denmark). Cell culture metabolites were measured using RX Imola (Randox Laboratories Ltd. Crumlin, UK) and osmolality was measured using a micro-Osmometer 2020 (Advanced Instruments, Norwood, MA).

Antibody titer was analyzed using a mAb-specific reverse-phase high-performance liquid chromatography (HPLC) or analytical Protein A HPLC method. Multiple product quality attributes were analyzed on harvested cell-culture fluid (HCCF) after a Protein A chromatography step. Analysis of HMW species was performed by size-exclusion chromatography, analysis of charge variants was performed by ion exchange chromatography, and analysis of N-glycan was performed by ultra-performance liquid chromatography (UPLC) with 2-aminobenzamide (2-AB) labeling.

Higher concentrations of components 9 and 12 had a significant negative impact on growth, viability and titer for all cell lines, and hence were removed from further analysis. Though not as severe as 9 and 12, higher concentration of component 16 also had impacted the cell culture performance for all cell lines, and higher concentrations of components 7 and 8 mostly affected mAb C. Components 7, 8 and 16 were included in further analysis for product quality. Cell culture performance parameters are presented below in Table 2, Table 3 and

TABLE 2
Cell culture performance for the cell line expressing mAb A

| | Relative IVCC | | Relative Viability | | Relative Titer | |
|---|---|---|---|---|---|---|
| | (% to Control) | | (% to Control) | | (% to Control) | |
| | Lower | Higher | Lower | Higher | Lower | Higher |
| | Concen- | Concen- | Concen- | Concen- | Concen- | Concen- |
| Component | tration | tration | tration | tration | tration | tration |
| 1 | 106.27 | 105.94 | 97.66 | 109.8 | 97.43 | 92.72 |
| 2 | 115.58 | 114.39 | 97.9 | 93.93 | 97.26 | 97.84 |
| 3 | 99.44 | 106.95 | 102.12 | 91.19 | 95.74 | 84.95 |
| 4 | 102.97 | 101.01 | 103.52 | 109.24 | 97.14 | 90.7 |
| 5 | 104.95 | 100.26 | 93.67 | 84.82 | 102.03 | 107.98 |
| 6 - GMP | 100.61 | 95.08 | 96.75 | 91.8 | 101.88 | 96.14 |
| 7 | 108.39 | 103.5 | 99.73 | 96.56 | 102.32 | 92.92 |
| 8 | 98.86 | 95.19 | 100.82 | 100.02 | 100.21 | 94.85 |
| 9 | 100.79 | 53.23 | 100.24 | 0.57 | 84.82 | 69.79 |
| 10 | 92.4 | 86.91 | 98.82 | 92.89 | 97.49 | 94.2 |
| 11 | 85.88 | 94.38 | 98.77 | 100.19 | 100.97 | 100.11 |
| 12 | 76.29 | 64.76 | 77.8 | 7.65 | 76.8 | 51.31 |
| 13 | 98.9 | 97.73 | 85.86 | 97.24 | 105.99 | 100.48 |
| 14 | 98.32 | 97.9 | 96.24 | 95.28 | 102.63 | 101.32 |
| 15 | 97.5 | 100.13 | 89.96 | 98.73 | 103.41 | 107.83 |
| 16 | 78.88 | 58.36 | 72.34 | 58.34 | 75.59 | Not |
| | | | | | | Available |
| 17 | 104.28 | 89.7 | 96.19 | 99.85 | 94.23 | 92.23 |
| 18 | 103.57 | 104.79 | 91.44 | 91.3 | 96.08 | 90.4 |

TABLE 3
Cell culture performance for the cell line expressing mAb B

| | Relative IVCC | | Relative Viability | | Relative Titer | |
|---|---|---|---|---|---|---|
| | (% to Control) | | (% to Control) | | (% to Control) | |
| | Lower | Higher | Lower | Higher | Lower | Higher |
| | Concen- | Concen- | Concen- | Concen- | Concen- | Concen- |
| Component | tration | tration | tration | tration | tration | tration |
| 1 | 107.66 | 101.84 | 101.9 | 104.31 | 97.69 | 85.29 |
| 2 | 102.94 | 99.61 | 101.19 | 96.07 | 99.08 | 95.18 |
| 3 | 95.94 | 94.07 | 101.54 | 99.37 | 98.69 | 45.74 |
| 4 | 97.43 | 97.26 | 101.7 | 100.68 | 98.47 | 96.98 |
| 5 | 98.28 | 96.19 | 102.81 | 99.85 | 99.57 | 90.88 |
| 6 - GMP | 96.14 | 90.77 | 100.77 | 95.28 | 100.81 | 94.86 |
| 7 | 95.55 | 75.67 | 98.33 | 33.2 | 98.39 | 68.69 |
| 8 | 96.67 | 76.9 | 98.6 | 36.93 | 98.3 | 65.87 |
| 9 | 96.52 | 62.79 | 98.54 | 1.13 | 70.32 | 40.32 |
| 10 | 101.29 | 97.3 | 107.73 | 101.65 | 103.33 | 101.07 |
| 11 | 101.81 | 100.01 | 107.09 | 105.27 | 104.34 | 100.61 |
| 12 | 93.72 | 75.8 | 100.18 | 6.79 | 100.19 | 48.41 |
| 13 | 100.11 | 97.58 | 98.09 | 97.91 | 102.75 | 101.65 |
| 14 | 97.94 | 100.2 | 97.7 | 99.72 | 98.73 | 101.19 |
| 15 | 95.89 | 99.22 | 100.06 | 98.21 | 100.81 | 102.99 |
| 16 | 94.03 | 64.78 | 75.5 | 64.47 | 116.2 | Not |
| | | | | | | Available |
| 17 | 106.24 | 104.72 | 108.02 | 106.92 | 137.22 | 140.38 |
| 18 | 98.93 | 99.92 | 99.69 | 101.3 | 134.72 | 138.76 |

TABLE 4

| | Relative IVCC | | Relative Viability | | Relative Titer | |
|---|---|---|---|---|---|---|
| | (% to Control) | | (% to Control) | | (% to Control) | |
| | Lower | Higher | Lower | Higher | Lower | Higher |
| | Concen- | Concen- | Concen- | Concen- | Concen- | Concen- |
| Component | tration | tration | tration | tration | tration | tration |
| 1 | 100.25 | 87.44 | 100.03 | 99.92 | 96.6 | 86.48 |
| 2 | 98.36 | 95.61 | 99.67 | 98.68 | 102.09 | 100.36 |
| 3 | 99.34 | 96.27 | 99.3 | 99.52 | 100.41 | 96.85 |
| 4 | 100.63 | 97.01 | 99.44 | 97.73 | 99.97 | 97.07 |
| 5 | 102.64 | 93.03 | 98.79 | 99.82 | 101.11 | 96.42 |
| 6 - GMP | 97.24 | 79.88 | 99.25 | 87.81 | 102.19 | 103.8 |
| 7 | 90.58 | 46.1 | 95.37 | 34.31 | 103.69 | 40.65 |
| 8 | 93.8 | 46.78 | 95.67 | 42.23 | 101.71 | 42.09 |
| 9 | 86.94 | 46.67 | 94.16 | 25.39 | 79.63 | 40.05 |
| 10 | 93.7 | 87.22 | 98.14 | 93.56 | 96.37 | 90.87 |
| 11 | 96.59 | 93.63 | 99.37 | 98.54 | 100.38 | 96.66 |
| 12 | 48.62 | 45.96 | 88.16 | 75.89 | 42.31 | 38.64 |
| 13 | 99.06 | 100.13 | 97.9 | 100.49 | 101.23 | 94.65 |
| 14 | 95.68 | 99.34 | 94.25 | 96.78 | 101.93 | 94.76 |
| 15 | 100.19 | 100.35 | 98.23 | 99.47 | 95.86 | 99.1 |
| 16 | 72.04 | 45.09 | 76.79 | 38.4 | 78.77 | Not |
| | | | | | | Available |
| 17 | 100.58 | 104.77 | 99.39 | 98.44 | 84.63 | 91.28 |
| 18 | 104.62 | 96.81 | 99.75 | 99.38 | 84.69 | 81.79 |

Data generated from all 6 blocks for all tested components (excluding component 9 and 12) were transformed using a dimensionless number, which is termed as Concentration Impact Factor, and abbreviated as C_{f}. C_{f} provides a score value for impact of change in component's concentration on desired response when tested at different concentrations. The equation used to transform data to calculate C_{f} is described below as equation 1. The first part of the equation represents a directional vector, which determines whether increasing the component concentration from a lower value to a higher value results in a positive or negative response for the measure attribute. The second part of the equation then calculates the magnitude of that change as the concentration changes from low to high.

$${C}_{f} = \{ \frac{H - L}{\vert H - L \vert} \} \cdot \{ \frac{H + L}{2 C} \cdot \frac{{h}_{c}}{{l}_{c}} \cdot \vert log \frac{H}{L} \vert \} \qquad ( equation 1 )$$

wherein,
C_{f}. Concentration impact factor (dimensionless number)
H: Response for a measured attribute when component is added at a higher concentration
L: Response for a measured attribute when component is added at a lower concentration
C: Response for a measured attribute when component is added at a lower concentration
h_{c}: high concentration (mM) of the component added
l_{c}: low concentration (mM) of the component added

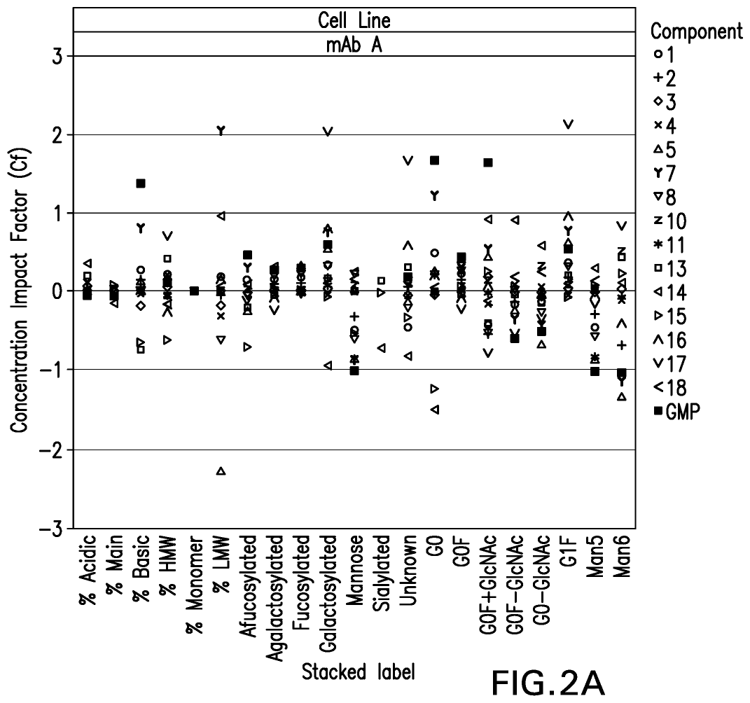

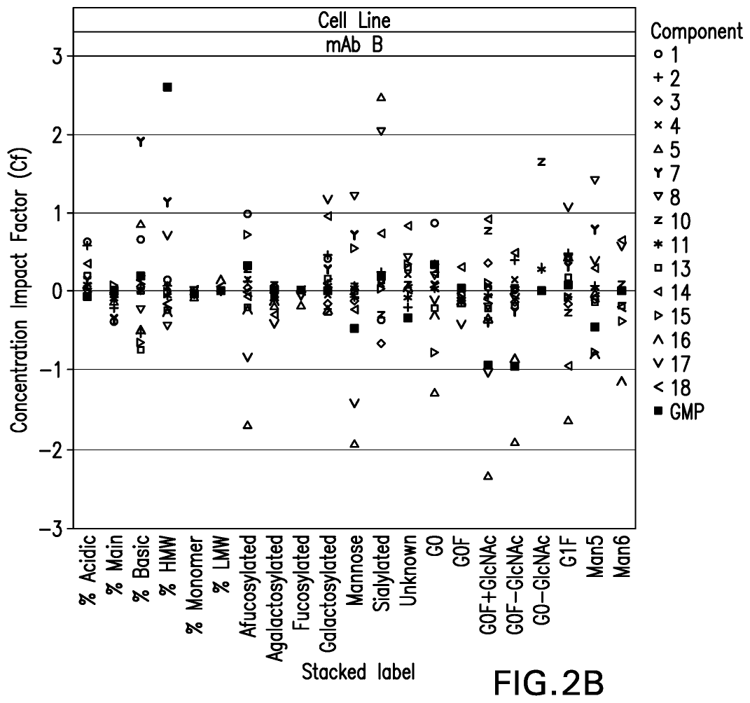

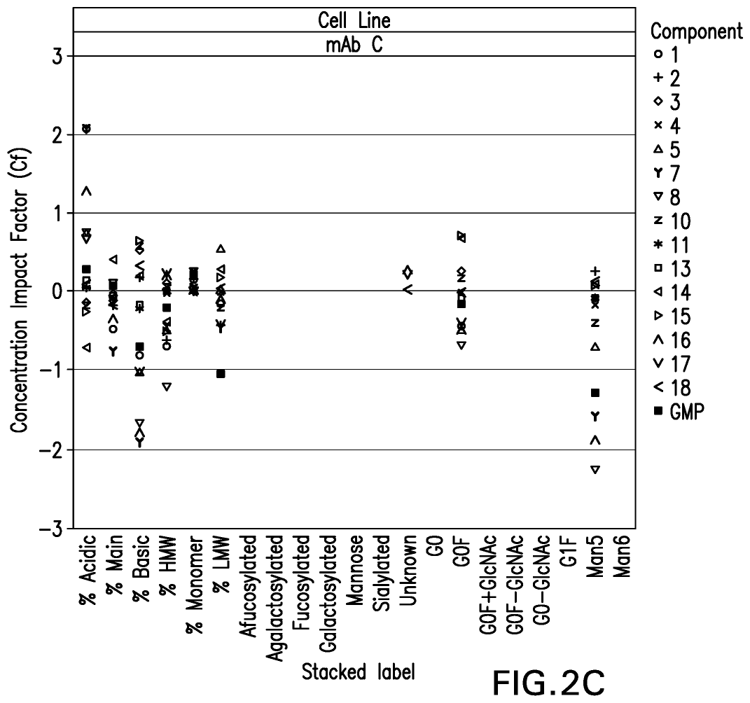

C_{f} was calculated for the aggregated species, charge variants and glycosylated species for cell lines expressing mAb A (FIG. 2A), mAb B (FIG. 2B), or mAb C (FIG. 2C). For mAb A, GMP resulted in the largest dose dependent decrease in Man 5 peak followed by components 5, 7 and 8. Components 15, 16 demonstrated slightly higher decrease in Man 5 peak than GMP for mAb B. Lastly for mAb C, components 7, 8 and 16 results in larger Man 5 decrease than GMP. However, components 7, 8 and 16 had a significant negative impact on the cell culture performance, and component 5 had a significant impact on other glycan species for mAb B. Hence components were not selected. Also, other components had a variety of impact on aggregation, charge variant, and glycan species.

Taken together, these results indicated that GMP has a potential to be used to decrease high mannose glycans on recombinant therapeutic antibodies with no negative impacts on product yield and other product quality attributes, including other glycosylated species.

### Example 2. Effect of GMP on HMG Content of Proteins and Cell Culture Parameters

The effect of GMP on recombinant CHO cell lines expressing monoclonal antibodies (mAb A, mAb B, or mAb C) that exhibit low, medium, or high levels of HMG content, respectively, was assessed in a 10-day fed-batch assay. Cells were cultured in Merck proprietary chemically defined media in increasing volume to obtain sufficient number of cells for inoculation. For cell lines expressing mAb A and mAb B, cells were seeded at 0.5×10⁶ cells per mL into Merck proprietary chemically defined production medium in two 1 L Erlenmeyer flasks at a final volume of 250 mL in each flask. For cell line expressing mAb C, cells were seeded at 1.0×10⁶ cells per mL into production medium in two 1 L Erlenmeyer flasks at a final volume of 250 mL in each flask. Cells were incubated at 36.5° C., with agitation at 100 rpm, in presence of 5% CO2, and humidity ≥70%. Merck proprietary chemically defined nutrient feed media were added to all the cultures on day 3, day 6, and day 8. On Day 6, cells from both 1 L flasks for each cell line were pooled and then aliquoted in 125 mL Erlenmeyer flasks with 30 mL working volume, respectively. A 200 mM stock solution of GMP was prepared by adding Guanosine 5′-monophosphate disodium salt hydrate (Sigma Aldrich, St. Louis, MO) in purified water. After the cells were aliquoted in separate 125 mL test flasks on day 6, GMP was added to the cultures at final concentration of 1 mM or 10 mM. No change in pH and osmolality was observed as a result of the GMP addition. A control condition with no added GMP was also created for each cell line. All conditions were run with two replicates. All cultures were harvested on day 10.

Cell culture parameters were analyzed on day 0, 3, 6, 8 and 10, and the corresponding spent medium supernatants were evaluated for antibody titer, charge variants, and glycan analysis on day 10 to assess the effect of GMP on cell growth, viability, titer, and glycan profile of the recombinant antibodies. Cell density and viability were measured using the trypan blue exclusion method with Cedex Bio Analyzer (Roche Diagnostics GmbH, Mannheim, Germany). Culture pH was measured using ABL80 Blood/Gas Analyzer (Radiometer, Denmark). Cell culture metabolites were measured using RX Imola (Randox Laboratories Ltd. Crumlin, UK) and osmolality was measured using a micro-Osmometer 2020 (Advanced Instruments, Norwood, MA).

Antibody titer was analyzed using a mAb-specific reverse-phase high-performance liquid chromatography (HPLC) or analytical Protein A HPLC method. Multiple product quality attributes were analyzed on harvested cell-culture fluid (HCCF) after a Protein A chromatography step. Analysis of HMW species was performed by size-exclusion chromatography, analysis of charge variants was performed by ion exchange chromatography, and analysis of N-glycan was performed by ultra-performance liquid chromatography (UPLC) with 2-aminobenzamide (2-AB) labeling.

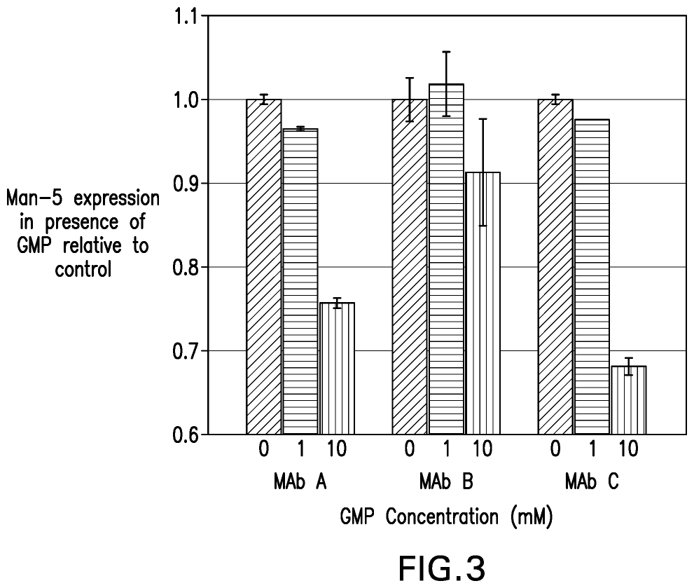

GMP caused a dose dependent decrease in HMG content on the recombinant antibodies as shown in FIG. 3 and Table 5. Man 5 was the major high mannose species downregulated upon GMP treatment. A slight decrease in higher order mannose structures was observed as well. At GMP concentration of 1 mM, there was no impact on cell culture parameters and slight decrease in high mannose glycans for mAb A and mAb C. At high concentration of 10 mM, GMP caused large decreases in high mannose in all cell lines, and slight decrease in cell growth for mAb A and mAb B, and considerable decrease in cell growth for mAb C. Titer and other product quality attributes (such as charge variants and aggregation) were largely unaffected for all cell lines. Each value is an average of duplicates.

TABLE 5
HMG levels (values relative to control)
for each cell line with GMP

| | GMP final | | |
|---|---|---|---|
| Cell Line | concentration (mM) | Man 5 | Total HMG |
| mAb A | 0 | 1 | 1 |
| | 1 | 0.97 | 0.96 |
| | 10 | 0.76 | 0.76 |
| mAb B | 0 | 1 | 1 |
| | 1 | 1.02 | 1.01 |
| | 10 | 0.91 | 0.90 |
| mAb C | 0 | 1 | N/A |
| | 1 | 0.98 | N/A |
| | 10 | 0.68 | N/A |

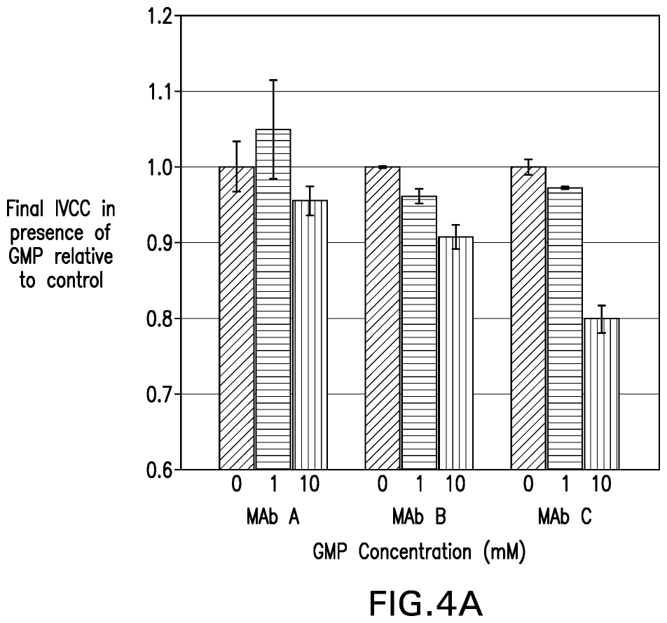

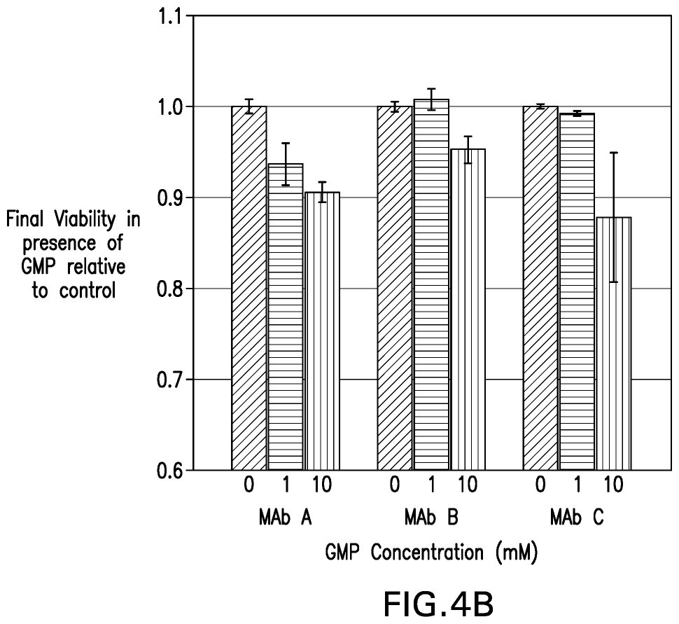

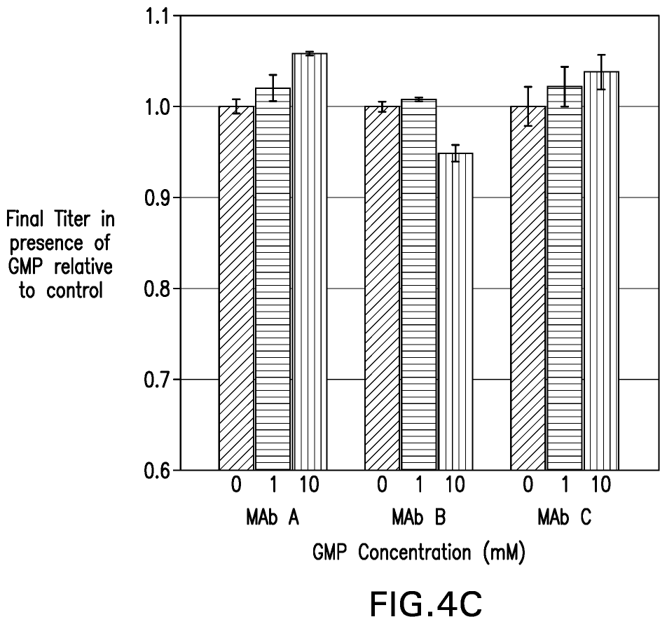

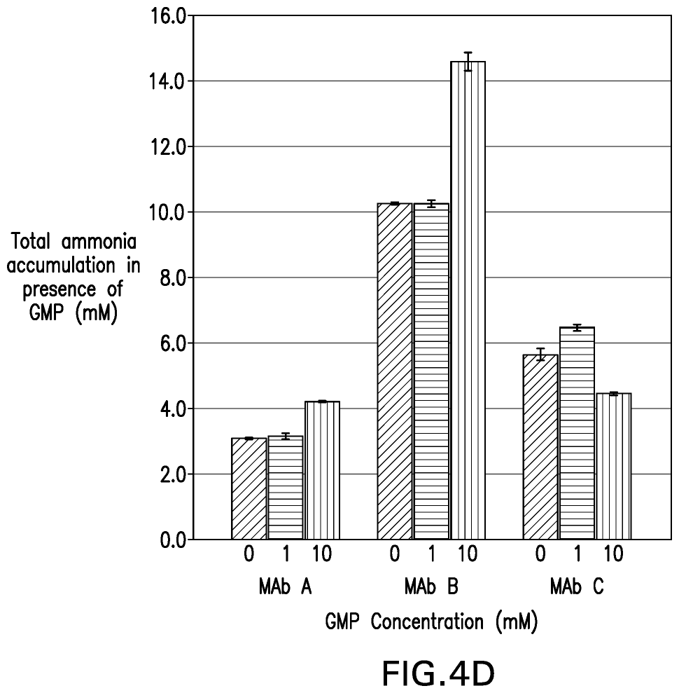

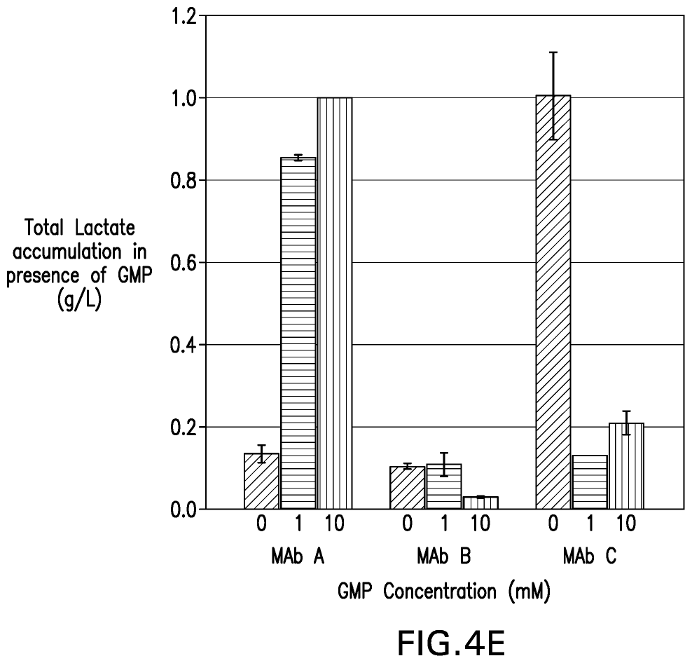

Cell culture performance was assessed via integral viable cell concentration (IVCC) (FIG. 4A), final viability (FIG. 4B), titer (FIG. 4C), ammonia (FIG. 4D), and lactate (FIG. 4E) measurements of harvested samples. Each value is an average of duplicates. The data are summarized in Table 6 below.

TABLE 6
Cell Culture Parameters

| | GMP final | IVCC | Final | | | Cumula- |
|---|---|---|---|---|---|---|
| | concen- | (10⁶ | via- | | | tive |
| Cell | tration | cells · | bility | Ammonia | Lactate | Titer |
| Line | (mM) | Day/mL) | (%) | (mM) | (g/L) | (mg/L) |
| mAb A | 0 | 44.2 | 91.2 | 10.3 | 0.11 | 155 |
| | 1 | 46.4 | 85.5 | 10.2 | 0.11 | 158 |
| | 10 | 44.3 | 77.4 | 14.6 | 0.03 | 167 |
| mAb B | 0 | 54.8 | 86.2 | 3.1 | 0.1 | 312 |
| | 1 | 52.7 | 86.9 | 3.2 | 0.9 | 314 |
| | 10 | 49.7 | 82.1 | 4.2 | 1.0 | 296 |
| mAb C | 0 | 88.9 | 98.3 | 5.6 | 1.1 | 676 |
| | 1 | 86.5 | 98.5 | 6.5 | 0.1 | 690 |
| | 10 | 71.1 | 86.3 | 4.5 | 0.2 | 701 |

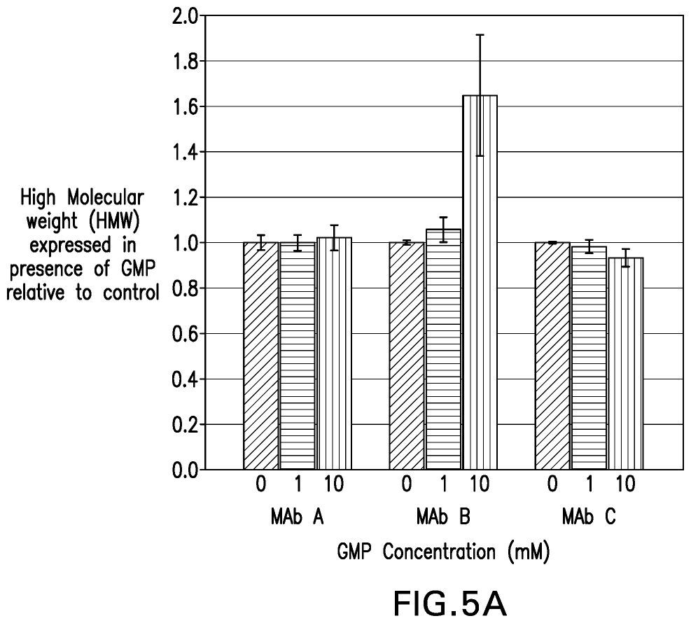

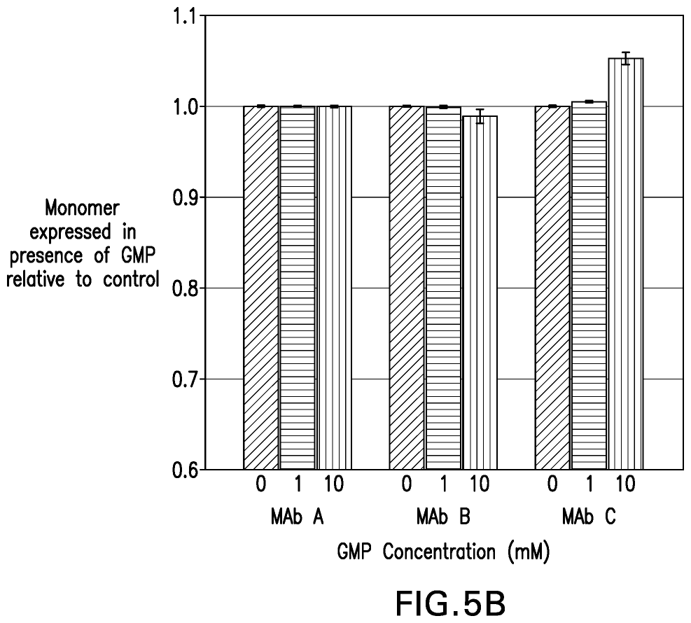

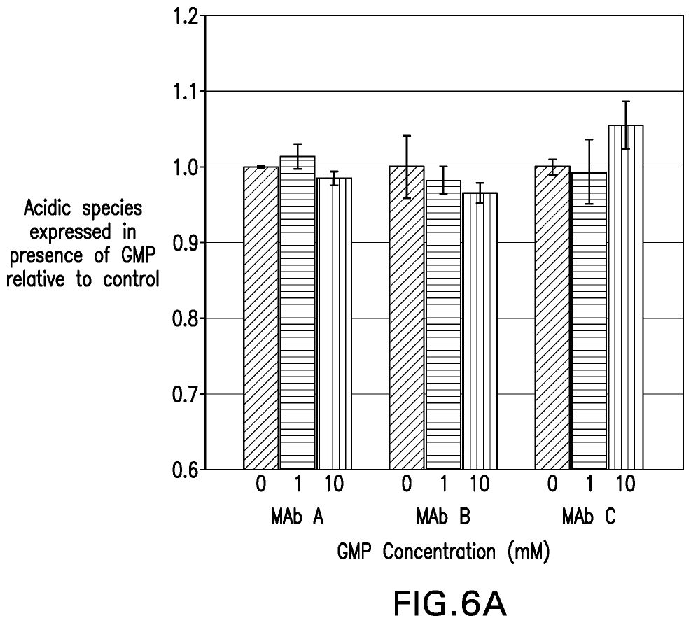

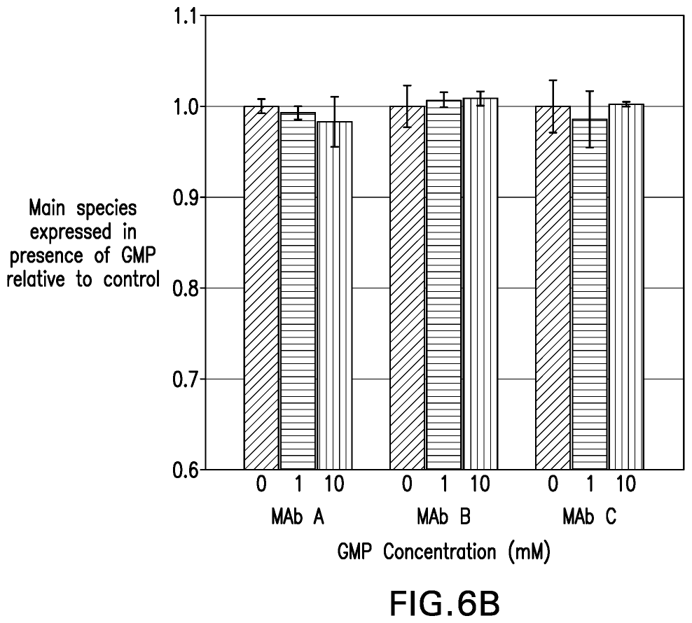

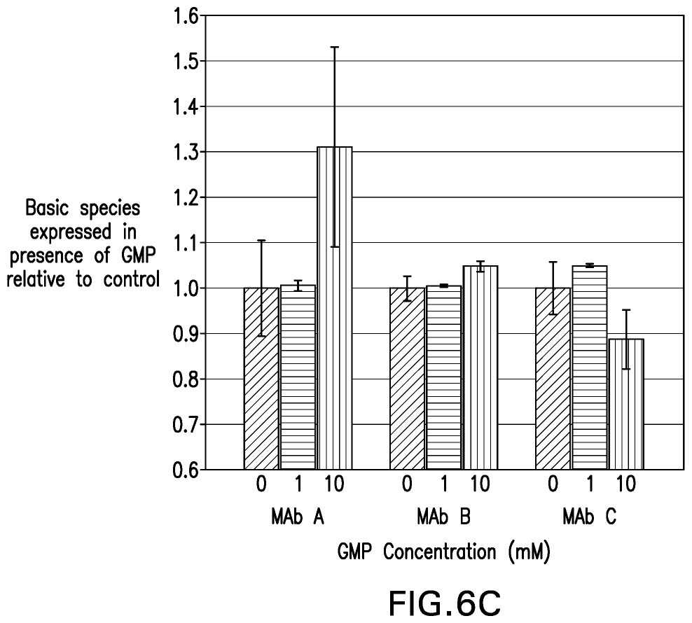

Other product quality attributes were assessed by measuring aggregation (FIGS. 5A and 5B) and charge variant species (FIGS. 6A-6C). Each value is an average of duplicates relative to respective control (0 mM GMP added condition). The data are summarized in Table 7 below.

TABLE 7
Product quality attributes (values relative to control)

| | | Total | | Total | | |
|---|---|---|---|---|---|---|
| | GMP final | Acidic | | Basic | High | |
| | concen- | Charge | Main | Charge | Molecular | |
| Cell | tration | Variants | Peak | variants | Weight | Monomer |
| Line | (mM) | (%) | (%) | (%) | (%) | (%) |
| mAb A | 0 | 1 | 1 | 1 | 1 | 1 |
| | 1 | 1.01 | 0.99 | 1.01 | 1.0 | 1.0 |
| | 10 | 0.99 | 0.98 | 1.31 | 1.02 | 1.0 |
| mAb B | 0 | 1 | 1 | 1 | 1 | 1 |
| | 1 | 0.98 | 1.01 | 1.01 | 1.06 | 1.0 |
| | 10 | 0.97 | 1.01 | 1.05 | 1.65 | 0.99 |
| mAb C | 0 | 1 | 1 | 1 | 1 | 1 |
| | 1 | 0.99 | 0.99 | 1.05 | 0.98 | 1.0 |
| | 10 | 1.06 | 1.00 | 0.89 | 0.93 | 1.05 |

Taken together, these results demonstrated that GMP decreases HMG on recombinant monoclonal antibodies with no negative impacts on product yield.

### Example 3. Effect of Various Agents that Increase GMP De Novo Synthesis on HMG Content of Proteins and Cell Culture Parameters

The effect of various agents that can increase GMP de novo synthesis when added to a mammalian cell culture are assessed in a fed-batch assay using at least one monoclonal antibody producing recombinant CHO cell line. These components are Inosine 5′-monophosphate (IMP), Xanthine 5′-monophosphate (XMP), Guanosine 5′-diphosphate (GDP), Guanosine 5′-triphosphate (GTP), Guanosine 3′, 5′-cyclic monophosphate (cGMP), Guanine, IMP dehydrogenase, and GMP Synthase. These components are assessed individually or in various combinations, and at several different concentrations to evaluate the impact of these agents on the HMG content of the mAb.

In addition to the HMG, other quality attributes, such as aggregation, acidic and basic charge variants, and cell culture performance parameters are evaluated. The experiments are conducted in experimental blocks and include appropriate control conditions. The experiments are executed as 10-day fed-batch or 14-day fed batch cultures. Additions of various agents at different stages of cell culture are also studied.

All references cited herein are incorporated by reference to the same extent as if each individual publication, database entry, patent application, or patent, was specifically and individually indicated to be incorporated by reference. This statement of incorporation by reference is intended by Applicant, pursuant to 37 C.F.R. § 1.57(b)(1), to relate to each and every individual publication, database entry, patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the embodiments in addition to those described herein will become apparent to those skilled in the art from the foregoing description and the accompanying figures. Such modifications are intended to fall within the scope of the appended claims.

1. A method of reducing a high mannose glycan (HMG) content of a protein expressed during a mammalian cell culture process, comprising:
(1) establishing a mammalian cell culture expressing the protein; and
(2) contacting the cell culture with Guanosine 5′-monophosphate (GMP) or an agent that increases GMP de novo synthesis,
wherein the HMG is Man 5.

2.-3. (canceled)

4. The method of claim 1, wherein step (2) comprises contacting the cell culture with GMP.

5. The method of claim 1, wherein step (2) comprises contacting the cell culture with an agent that increases GMP de novo synthesis, wherein the agent is Inosine 5′-monophosphate (IMP), Xanthine 5′-monophosphate (XMP), Guanosine 5′-diphosphate (GDP), Guanosine 5′-triphosphate (GTP), Guanosine 3′, 5′-cyclic monophosphate (cGMP), Guanine, IMP dehydrogenase, GMP synthase, or a combination thereof.

6.-8. (canceled)

9. The method of claim 4, wherein the final concentration of GMP in the cell culture is from 1 mM to 25 mM.

10.-13. (canceled)

14. The method of claim 4, wherein the GMP is added to the cell culture between 3 and 15 days after the cell culture is established.

15.-16. (canceled)

17. The method of claim 1, wherein the cell culture is maintained by perfusion for a partial or entire period of the cell culture.

18.-19. (canceled)

20. The method of claim 1, wherein the cell culture is maintained by fed batch for a partial or entire period of the cell culture.

21.-22. (canceled)

23. The method of claim 1, wherein the cell culture is maintained by a combination of perfusion and fed batch for a partial or entire period of the cell culture.

24. The method of claim 1, wherein the HMG content of the protein is reduced by at least 1%, compared to the HMG content of the protein expressed in an essentially same cell culture except that the essentially same cell culture is not contacted with the GMP or the agent that increases GMP de novo synthesis.

25.-26. (canceled)

27. The method of claim 24, wherein the HMG content of the protein is reduced by at least 10%.

28. The method of claim 24, wherein the HMG content of the protein is reduced by at least 20%.

29. The method of claim 24, wherein the HMG content of the protein is reduced by at least 30%.

30.-31. (canceled)

32. A cell culture medium for reducing HMG content of a protein expressed during a mammalian cell culture process, comprising GMP or an agent that increases GMP de novo synthesis, wherein the GMP or the agent that increases GMP de novo synthesis is at a final concentration sufficient to reduce the HMG content of the protein by at least 1% compared to the protein expressed in an essentially same cell culture medium except that the essentially same cell culture medium does not include GMP or the agent that increases GMP de novo synthesis.

33. (canceled)

34. The cell culture medium of claim 32, wherein the cell culture medium comprises GMP, and wherein the GMP is at a final concentration of 1-25 mM.

35.-38. (canceled)

39. The cell culture medium of claim 32, wherein the cell culture medium comprises an agent that increases GMP de novo synthesis, wherein the agent is Inosine 5′-monophosphate (IMP), Xanthine 5′-monophosphate (XMP), Guanosine 5′-diphosphate (GDP), Guanosine 5′-triphosphate (GTP), Guanosine 3′, 5′-cyclic monophosphate (cGMP), Guanine, IMP dehydrogenase, GMP synthase, or a combination thereof.

40. (canceled)

41. The method of claim 1, wherein the mammalian cell culture is a CHO cell culture.

42. The cell culture medium of claim 32, wherein the cell culture medium is a CHO cell culture medium.

43. The method of claim 1, wherein the protein is a monoclonal antibody.

44. The cell culture medium of claim 32, wherein the protein is a monoclonal antibody.
